(19) United States
(12) Patent Application Publication
(10) Pub. No.: US 2006/0039887 A1
(43) Pub. Date: Feb. 23, 2006

(54) Cosmetic or pharmaceutical composition for skin care

(75) Inventors: Shyam K. Gupta, Scottsdale, AZ (US); Edward G. Hoyt, Fountain Hills, AZ (US)

Correspondence Address: THORPE NORTH & WESTERN, LLP., 8180 SOUTH 700 EAST, SUITE 200, SANDY, UT, 84070, US

(73) Assignee: Infinity2 Health Sciences, Inc.

(21) Appl. No.: 11/208,306

(22) Filed: Aug. 18, 2005

Related U.S. Application Data
(60) Provisional application No. 60/603,477, filed on Aug. 20, 2004.

(51) Int. Cl. A61K 8/97; A61K 36/28

(52) U.S. Cl. 424/74; 424/725

(57) ABSTRACT

The present invention relates to a cosmetic or pharmaceutical composition to reduce skin damage caused by aging and/or the environment. The composition can include a genus Centipeda plant extract, a trace metal source in a skin absorbing form in an amount effective for activating or enhancing superoxide dismutase enzyme, and a carrier suitable for topical administration. The compound may alternatively include a genus Centipeda plant extract and a low molecular weight transporter and an ion-pair delivery system including a donating composition and an accepting composition, wherein the donating composition and the accepting composition are combined to form a bound ion-pair, and a carrier suitable for topical administration.

The present application claims the benefit of U.S. Provisional Application No. 60/603,477 filed on Aug. 20, 2004, which is incorporated herein by reference in its entirety.

### FIELD OF THE INVENTION

The present invention relates generally to topical compositions that treat various skin conditions, and more particularly to compositions including extracts from the plant Centipeda cunninghamii.

### BACKGROUND OF THE INVENTION

The condition and appearance of the skin is a major factor in maintaining a youthful appearance, which is of great concern to many people, particularly in an aging population. As people grow older, they begin to experience wrinkles and fine lines, a slackening of tissue, a loss of cutaneous elasticity, and often a leathery or dry appearance to their skin. Also, the skin begins to take on a yellow appearance and loses its radiance. Age-spots are often characteristic, becoming pronounced on the face, neck, chest, and arms. Skin that has been consistently exposed to sunlight throughout life may show pigmentation marks, telangiectasia, and elastosis. Furthermore, at a histological level, skin damage from photo-aging can show tangled, thickened, and/or abnormal elastic fibers, decreased collagen, and/or increased glycosaminoglycan content. The aging process can also result in thinning and deterioration of the skin as well as hair loss. There can also be a reduction in cells and in blood supply, as well as a flattening in the junction between the dermis and epidermis.

Treatments designed to prolong or promote a youthful appearance often include topical applications of cosmetic preparations, lotions or moisturizers, electrical stimulation, collagen injections, and/or cosmetic surgery. However, despite all of these options and topically applied products available, there is still a serious need for skin care compositions that treat wrinkles and fine lines to restore the youthful appearance of the skin.

### SUMMARY OF THE INVENTION

It has been recognized that it would be advantageous to develop a topical composition that reduces skin damage caused by aging and/or environmental impact. Specifically, in one aspect of the present invention, a cosmetic or pharmaceutical composition can comprise a genus Centipeda plant extract, a trace metal source in a skin absorbing form in an amount effective for activating or enhancing superoxide dismutase enzyme activity, and a carrier suitable for topical administration.

In another aspect, a cosmetic or pharmaceutical composition can comprise a genus Centipeda plant extract, a copper source, and a carrier suitable for topical administration.

In yet another aspect of the present invention, a cosmetic or pharmaceutical composition can comprise a genus Centipeda plant extract, an ion-pair delivery system including a donating composition and an accepting composition, wherein the donating composition and the accepting composition are combined to form a bound ion-pair, and a carrier suitable for topical administration.

In another embodiment, a method of reducing the appearance of aging of a skin surface can comprise topically applying a cosmetic or pharmaceutical composition to a skin surface. The composition can comprise a genus Centipeda plant extract, a carrier suitable for topical administration, and at least one additional component. The at least one additional component can be selected from the group consisting of (a) a trace metal source in a skin absorbing form in an amount effective for activating or enhancing superoxide dismutase enzyme, (b) a copper source, and (c) an ion-pair delivery system including a donating composition and an accepting composition, wherein the donating composition and the accepting composition are combined to form a bound ion-pair.

Additional features and advantages of the invention will be apparent from the following detailed description which illustrates, by way of example, features of the invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a carrier” includes reference to one or more of such materials.

As used herein, the term “apoenzyme” refers to an enzyme that is not in its active form. A combination of an apoenzyme with a cofactor, such as a trace metal, converts the apoenzyme into a functional enzyme.

As used herein, the term “dispersion” refers to an emulsion or suspension comprising a dispersed substance and the medium it is dispersed in.

As used herein, the term “emulsion” refers to a mixture of two incompletely miscible liquids. Typically, an emulsion includes a continuous phase and a discontinuous phase.

As used herein, the term “low molecular weight” (LMW) refers to molecules of 3000 Dalton units (Da) or less that can pass through a typical dialysis membrane. In a preferred embodiment, LMW can be less than about 1000 Da.

As used herein, the term “parts per million” (ppm) refers to the number of parts of a material or molecule in one million parts of a composition. For example, if 1 wt % copper gluconate is added to a composition, then that composition contains 10,000 parts by weight of copper gluconate (or 1,400 ppm by weight of copper ions, as copper gluconate includes 14 wt % copper) in one million parts of that composition.

Reference to signs of “skin aging” or “aging of a skin surface” includes all outward visibly and tactilely perceptible manifestations as well as any other macro or micro effects due to skin aging. Such signs may be induced or caused by intrinsic factors or extrinsic factors (e.g., chronological aging and/or environmental damage, including damage from pollution, the sun, burns, injury resulting in skin abrasion, etc.). These signs may result from processes which include, but are not limited to, the development of textural discontinuities such as wrinkles and coarse deep wrinkles, skin lines, crevices, bumps, large pores (e.g., associated with adrenal structures such as sweat gland ducts, sebaceous glands, or hair follicles), unevenness or roughness, loss of skin elasticity (loss and/or inactivation of functional skin elastin), sagging (including loss and/or damage to functional subcutaneous muscle tissue and including puffiness in the eye area and jowls), loss of skin firmness, loss of skin tightness, loss of skin recoil from deformation, discoloration (including under eye circles), blotching, shallowness, hair loss, hyper pigmented skin regions such as age spots and freckles, keratoses, abnormal differentiation, hyperkeratinization, elastosis, collagen breakdown, or other histological changes in the stratum corneum, dermis, epidermis, the skin vascular system (e.g., telangiectasia or spider vessels), or underlying tissues, especially those proximate to the skin.

As used herein, the term “trace metal(s)” refers to transition metal(s) found in very small amounts in plant and animal tissues which have an effect upon a biochemical process. Trace metals can include ferrous and ferric iron, copper, zinc, manganese, chromium, molybdenum, selenium, etc., and typically exclude alkali metals and alkaline earth metals, with the exception of magnesium in accordance with embodiments of the present invention. Trace metals are depleted through the expenditure of energy by a living organism, and are traditionally known to be replenished in animals by eating plants, animals, and other nutrients. In accordance with embodiments of the present invention, trace metals can be absorbed by the skin to provide anti-aging benefits.

With these definitions in mind, a cosmetic or pharmaceutical composition for resisting skin damage associated with aging or the environment is disclosed. In one aspect of the present invention, the composition can include a genus Centipeda plant extract, a trace metal source in a skin absorbing form, and a carrier suitable for topical administration. The trace metal source can be present in the composition in an amount effective for activating or enhancing superoxide dismutase (SOD) enzyme activity. The plant extract from a genus Centipeda plant can be from the plant Centipeda cunninghamii. In one embodiment, the trace metal source can be any trace metal source known to be useful to one skilled in the art, including, but not limited to, copper, zinc, manganese, and combinations thereof.

Specifically, turning to one of the more beneficial trace metals, copper (optionally coadministered with zinc and/or manganese) can provide benefits for normal development and function of human cells. Copper is the third most abundant trace element in human body, with vitamin-like impact on living systems. Copper can function as a cofactor for at least 30 enzymes (or apoenzymes), and the ability of copper to cycle between oxidized Cu²⁺ and reduced Cu⁺ states can be used by cuproenzymes involved in redox reactions, two of the more important examples being Cu/Zn superoxide dismutase (SOD) and cytochrome C oxidase. Cu/Zn SOD is an enzyme responsible for the destruction of toxic superoxide anions in the human body that directly relates to the processes of skin aging.

As copper or other trace metals can be more beneficial for skin health when absorbed, a skin absorbing form of the trace metal source can be used. In one embodiment, such a composition can comprise a low molecular weight transporter composition coupled to the trace metal. More specifically, in certain embodiments, the low molecular weight transporter composition can be bound to the trace metal source, and can be a nucleotide, a phosphorylated saccharide, a phosphorylated glycoside, or a combination thereof. In one aspect, the low molecular weight transporter composition can be a nucleotide such as adenosine triphosphate (ATP), adenosine diphosphate (ADP), adenosine monophosphate (AMP), flavin adenine dinucleotide (FAD), guanosine monophbsphate (guanylic acid), guanosine diphosphate, inosine monophosphate (inosinic acid), inosine diphosphate, nicotinamide adenine dinucleotide (NAD), nicotinamide adenine dinucleotide reduced (NADH), citicholine, or a combination thereof. In another aspect, the low molecular weight transporter composition can be glucose-1-phosphate, glucose-6-phosphate, glucose-1,6-diphosphate, fructose-1-phosphate, fructose-6-phosphate, fructose-1,6-diphosphate, sucrose phosphate, or a combination thereof.

Alternatively, genus Centipeda plant extract can be coadministered with an ion-pair delivery system comprising a donating composition and an accepting composition. In this embodiment, the donating composition and the accepting composition are combined to form a bound ion-pair. In one aspect, the donating composition can be an electron donating composition and the accepting composition can be an electron accepting composition. In another aspect, the donating composition can be a proton donating composition and the accepting composition can be a proton accepting composition. In either aspect, it is preferred that the ion-pair provides improved bioavailability compared to each component alone, and dissociates into the donating composition and the accepting composition following absorption through the skin. The ion-pair can also dissociate into the donating composition and the accepting composition when the ion-pair is exposed to physiological pH conditions, for example. It is important to note that a few compositions can be considered to be both trace metals in skin absorbing forms as well as ion-pair-type compositions.

Whether a trace metal in a skin absorbing form, a copper source, or an ion-pair composition, or a combination of these compositions is administered to the skin to reduce the effects of aging or environmental skin damage, the present invention relates to the benefits of co-administration of one or more of these compositions with a genus Centipeda composition. Typically, the genus Centipeda composition will be administered in the form of a Centipeda cunninghamii extract, though this-is not required.

Genus Centipeda Composition

In one aspect of the present invention, a genus Centipeda plant composition can be co-administered with a number of other anti-aging compositions, as will be described in more detail herein. In one embodiment, an extract from the plant Centipeda cunninghamii can be included (or co-administered) in one of a few types of compositions (e.g., copper-containing compositions, skin-absorbable trace metal-containing compositions, and ion-pair-containing compositions, to name a few). When combined with a suitable carrier, this extract is useful for topical treatment or prophylaxis of a variety of medical conditions, including acne, eczema, dermatitis, psoriasis, skin irritations, bed sores, and other various skin conditions. It can also promote or increase the rate of skin cell renewal, and provide protection against damage caused by sunburn.

Irrespective of other materials that are present in the topical creams or ointments, etc., of the present invention, though any effective amount can be included, the genus Centipeda plant extract can be present at a concentration of from 0.0001% to 30.0 wt % of the cream, ointment or other composition to be applied. In a more specific embodiment, the extract can be present at from 0.01 wt % to 10.0 wt %, and in another embodiment, from 0.1 w% to 5.0 wt %.

Trace Metal Source

In one aspect of the present invention, the composition containing a Centipeda cunninghamii extract can also include a trace metal source. Though many trace metals are useful, in one embodiment, the trace metal can be copper. In another aspect, the trace metal can be zinc or manganese. In yet another aspect, multiple trace metals can be present, such as any combination of copper, zinc, and/or manganese.

One example of a process that utilizes trace metals in human physiology is superoxide dismutase (SOD). SOD is a metalloenzyme that can be linked with the process of aging and carcinogenesis in humans. SOD enzyme contains both copper and zinc at its active site. Using copper as an example, in a specific embodiment, several compositions can work together to deliver this or other trace metals to appropriate skin sites for proper cell function, as follows:
(i) a source of copper;
(ii) a transporter(s) of copper from extra-cellular to intra-cellular levels;
(iii) a storage molecule or device for copper within the cell;
(iv) a chaperone to transport copper from the storage molecule to the apoprotein of SOD enzyme;
(v) an energy source for the transport of copper from copper storage molecule to the apoprotein of SOD (which, in many cases, is copper ATPase); and
(vi) additional cofactor trace metals, such as zinc, manganese, and/or iron.

With this basic scheme as illustrating one embodiment of trace metal delivery to appropriate skin cell sites, trace metals such as copper, zinc, iron, and manganese that may be relevant for the proper functioning of SOD and other deactivators of active-oxygen molecules can be delivered via topical compositions. This can be achieved by the preparation of copper and other trace metal complexes with phosphorylated nucleosides and/or phosphorylated mono-saccharides, such as nucleotides and glycosides. These trace metal complexes of nucleotides or glycosides can be prepared by an in situ method in water, water soluble or miscible organic solvents, or a mixture of water and water soluble or miscible organic solvents in accordance with exemplary steps as follows.

A first step can include combining two or more water soluble trace metal donor derivatives that can be inorganic or organic in nature. Using copper as an example, copper chloride, copper sulfate, copper nitrate, copper amino acid chelate, copper EDTA, copper peptide, copper gluconate, and/or copper histidinate can be used in any combination. Other known derivatives of copper or other trace metals can also be used. To illustrate, a trace metal donor derivative can be combined with a trace metal transporter derivative, such as a nucleotide or a phosphorylated mono-saccharide (glycoside). Copper, or other trace metals, can be transferred from their inorganic or organic donor derivative to the phosphoric acid center of nucleotide or phosphorylated mono-saccharide. The nitrogen centers of nucleotide and hydroxyl centers of glycoside provides further chelating centers to stabilize such trace metal nucleotides or trace metal glycosides. Representative examples are shown in Equations 1 to 5, where trace metals are moved from trace metal donors to low molecular weight (LMW) transporter molecules, as shown below:
Cu-Gluconate+ATP+Cu-ATP+Gluconic Acid (Equation 1)
Cu-Histidinate+ADP+Cu-ADP+Histidine (Equation 2)
Cu-Gluconate+DNA+Cu-DNA+Gluconic Acid (Equation 3)
ZnCl₂+Na₂-Fructose-1,6-diphosphate→Zn (Fructose-1,6-diphosphate)+2 NaCl (Equation 4)
CuCl₂+Na₂-Fructose-1,6-diphosphate→Cu (Fructose-1,6-diphosphate)+2 NaCl (Equation 5)

A further step can include mixing the solution of the trace metal derivative of the nucleotide or the glycoside that is formed (such as in Equations 1-5) with a base of topical composition prepared separately. The composition formed can then be stored under ambient storage conditions for a later use, or alternatively, immediately used.

The exchange of the trace metal from a trace metal donor chelate (as in Equations 1-3) to a low molecular weight (LMW) transporter molecule is both surprising and unexpected, as chelating agents often do not tend to allow the migration of a metal bound thereto to another non-chelating molecule. Without being bound by any particular theory, the selection of a trace metal donor and the LMW transporter molecule is best achieved when the pH, pK, or pK₁ value of the acid part of the trace metal donor is higher than the pH, pK, or pK₁ of LMW transporter molecule. For example, copper-ATP can be made from ATP and copper gluconate (which is made from gluconic acid and a copper source). The pH of a 1 wt % solution of gluconic acid in water is 2.5. The pH of a 1 wt % solution of ATP in water is 2.0. Therefore, the pH of the gluconic acid solution is higher than the pH of the ATP solution. Subsequently, when a solution of copper gluconate in water is mixed with a solution of ATP in water, the copper from the gluconate moiety migrates to the ATP moiety to form copper-ATP. Copper-ATP is a LMW transporter of copper, whereas copper gluconate is not a LMW transporter of copper. In another embodiment, a 1 wt % solution of fructose-6-phospate has a pK of 1.2. The glycine moiety in copper glycinate has a pK of 2.34. Therefore, when a water solution of fructose-6-phosphate is mixed with a water solution of copper glycinate, which is a chelated form of copper, then copper migrates from glycine moiety to fructose phosphate moiety to form copper fructose-6-phosphate, a LMW transporter of copper. In a yet another example, the pK₁ of ascorbic acid is 4.17, and the pK₁ of glucose-1-phosphate is 1.11. Therefore, by mixing a water solution of zinc ascorbate and a water solution of glucose-1-phosphate, a water solution of zinc glucose-1-phosphate is obtained in situ.

The amount of trace metal delivered by the trace metal transporter for intracellular functions can vary significantly. This is because various trace metals can be utilized in vastly different amounts for such functions. For example, a human body of approximately 75 kilograms contains only about 250 milligrams, or 3 to 4 parts per million (ppm) of copper, whereas the human body contains about 2 to 3 grams or about 30 to 40 ppm of zinc. Because various delivery systems can have a profound effect on how much actual trace metal is delivered in vivo, it is difficult to exactly calculate how much trace metal is needed in a topical composition. However, this difficulty is reduced in the present invention because the exact nature and amount of a trace metal in a composition can be determined from the trace metal ingredients used in the preparation of that composition. It is thus possible to deliver 3 to 4 ppm of copper or 30 to 40 ppm of zinc in a predetermined amount, if so desired, by an in situ preparation method.

The intra-cellular storage molecule for trace metals can be a sulfur-containing molecule. Glutathione is an example of a useful composition, although other similar molecules such as N-acetyl cysteine, thioglycolic acid, and metallothionein can also be used. The amount of such storage molecules can be configured to be in proportion to the trace metal that is being delivered for intra-cellular functions. If an intra-cellular reservoir of storage molecules does not need any supplementation, then additional storage molecules may not be necessary in the formulation.

Further, both the energy source required for the transport of trace metal from the storage molecule to SOD apoenzyme and the trace metal transporter molecule for the transport of trace metal from skin surface to deeper layers of skin can be the same molecule. For example, ATP, ADP, fructose-1,6-diphosphate, and glucose phosphate can perform this dual function of being the transporter of trace metals through dermal layers, as well as be the provider of intra-cellular energy source required for the transport of trace metals from their storage molecule to the apoenzyme.

Irrespective of other materials that are present in the topical compositions of the present invention, though any effective amount can be included, the trace metal(s) can be delivered by the (LMW) transporter molecule in an amount from 0.0001 wt % to 10.0 wt %, and in another embodiment, from 0.1 w% to 1.0 wt % in metal content.

Other information related to delivery of trace metals to the skin is disclosed in U.S. patent application No. 2004/0105894, which is incorporated herein by reference in its entirety.

Ion-Pair Delivery

In one aspect of the present invention, the cosmetic or pharmaceutical composition of the present invention can include an ion-pair mechanism in which one composition is an electron-donor or proton-acceptor, and the other composition is an electron-acceptor or proton-donor. These compositions can combine to form ion-pair combinations that can be more bioavailable when applied to the skin, have better stability, and are economical to produce from commonly available ingredients. The ion-pair composition can dissociate into the donating composition and the accepting composition following absorption through the skin, or when the ion-pair composition is exposed to physiological pH conditions. As mentioned, these compositions can be co-administered with a genus Centipeda plant extract in accordance with embodiments of the present invention.

The ion-pair compositions formed are not merely the mixtures of the two compositions that are combined in an ion-pair mode. Such ion-pair compositions are discreet chemical entities. For example, the combination of niacinamide with ascorbic acid results in the formation of ion-pair complex, niacinamide ascorbate. In this example, niacinamide, which is a proton-acceptor composition, has the following properties: crystalline white powder, melting point 130° C., pH of 1% water solution=6.3, solubility in water=50%, stability of water solution=good. Ascorbic acid, which is a proton-donor composition, has the following properties: crystalline white powder, melting point 162° C., pH of 1% water solution=2.6, solubility in water=25%, stability of water solution=poor. Niacinamide ascorbate, the ion-pair that is produced from the combination of the above two compositions has the following properties: crystalline yellow powder, melting point 142° C., pH of 1% water solution=3.7, solubility in water=40%, stability of water solution=good. Niacinamide ascorbate is rapidly absorbed into skin from water solutions, and upon reaching physiological pH conditions beneath the skin surface, the ion-pair composition undergoes ionic separation to release both niacinamide and ascorbic acid in their original molecular state.

Ascorbic acid is known to be a problematic ingredient in cosmetic compositions from a stability and bioavailability point of view. However, the ion-pair compositions formed from the combination of ascorbic acid with appropriate electron-donor compositions are more stable and are easier to formulate in such cosmetic compositions. Other examples of such ion-pair compositions of ascorbic acid include, but not limited to, glucosamine ascorbate, arginine ascorbate, lysine ascorbate, glutathione ascorbate, nicotinamide ascorbate, niacin ascorbate, allantoin ascorbate, creatine ascorbate, creatinine ascorbate, chondroitin ascorbate, chitosan ascorbate, DNA ascorbate, and carnosine ascorbate.

Hydroxycitric acid is a popular composition for weight loss management. However, it is unstable in its free acid form and is known to undergo cyclication reaction to form Garcinia acid, which does not have weight loss benefits. Most preparations of hydroxycitric acid are thus based on its alkali and alkaline earth metal salts, such as tri-potassium hydroxycitrate. In such compositions, the tri-potassium part does not provide any weight loss benefits. However, by ion-pair combination of hydroxycitric acid with niacinamide, niacinamide hydroxycitrate ion-pair is obtained in which both hydroxycitric acid part and niacinamide part provide slimming benefits. Moreover, hydroxycitric acid part is not cyclized to Garcinia acid form. Additional skin beneficial ion-pair compositions of hydroxycitric acid (HCA) can also be prepared, for example Allantoin HCA, Glucosamine HCA, Creatine HCA, Carnitine HCA, Niacinamide HCA, Pyridoxine HCA, Chitosan HCA, Niacin HCA, Benzyl Niacin HCA, Methyl Niacin HCA, Caffeine HCA, Aminophylline HCA, Chromium picolinate HCA, Phaseolamin HCA, Theophylline HCA, Theobromine HCA, Synephrine HCA, Hordenine HCA, Octopamine HCA, Tyramine HCA, and N-Methyltyramine HCA, and the like.

AHA (alpha-hydroxy acids) and BHA (beta-hydroxy acids) are very popular cosmetic compositions that provide skin rejuvenating benefits. However, such acids are known to cause skin irritation. If they are neutralized with an alkali, for example, then skin irritation problems are reduced, but their skin rejuvenating efficacy is also significantly reduced or even eliminated. The combination of AHA and BHA with proton-accepting compositions can eliminate or reduce these problems, and such ion-pair compositions, for example allantoin lactate, allantoin glycolate, allantoin mandelate, allantoin malate, allantoin ascorbate, allantoin phytate, allantoin citrate, allantoin hydroxy citrate, allantoin aleurate, allantoin salicylate, allantoin hyaluronate, glucosamine lactate, glucosamine glycolate, glucosamine malate, glucosamine mandelate, glucosamine ascorbate, glucosamine phytate, glucosamine citrate, glucosamine hydroxy citrate, glucosamine aleurate, glucosamine salicylate, glucosamine hyaluronate, creatine lactate, creatine glycolate, creatine malate, creatine mandelate, creatine ascorbate, creatine phytate, creatine citrate, creatine hydroxy citrate, creatine aleurate, creatine salicylate, creatine hyaluronate, niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aleurate, pyridoxine salicylate, pyridoxine hyaluronate, chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, and chitosan hyaluronate can be composed for their various skin and body beneficial applications.

To exemplify a specific skin care embodiment, one can consider the treatment of acne. Several acne compositions are also known. Such compositions can be further enhanced in their efficacy and bioavailability by ion-pair combination process described in the present invention. The examples of such acne compositions include, but not limited to, niacinamide salicylate, niacinamide ascorbate, niacinamide folate, niacinamide lipoate, niacinamide lactate, niacinamide glycolate, niacinamide mandalate, niacinamide malate, niacinamide hydroxycitrate, niacinamide hydroxytetronate, niacinamide aleurate, niacinamide petroselinate, niacinamide pantothenate, niacinamide adenosine monophosphate (AMP), niacinamide diphosphate (ADP), niacinamide adenosine triphosphate (ATP), niacinamide hydroquinone carboxylate, allantoin lactate, allantoin glycolate, allantoin mandelate, allantoin malate, allantoin ascorbate, allantoin phytate, allantoin citrate, allantoin hydroxy citrate, allantoin aleurate, allantoin salicylate, allantoin hyaluronate, glucosamine lactate, glucosamine glycolate, glucosamine malate, glucosamine mandelate, glucosamine ascorbate, glucosamine phytate, glucosamine citrate, glucosamine hydroxy citrate, glucosamine aleurate, glucosamine salicylate, glucosamine hyaluronate, creatine lactate, creatine glycolate, creatine malate, creatine mandelate, creatine ascorbate, creatine phytate, creatine citrate, creatine hydroxy citrate, creatine aleurate, creatine salicylate, creatine hyaluronate, niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aleurate, pyridoxine salicylate, pyridoxine hyaluronate, chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate, azelaic acid, niacinamide azelate, pyridoxine azelate, chitosan azelate, glucosamine azelate, retinoic acid, niacinamide retinoate, pyridoxine retinoate, chitosan retinoate, and glucosamine retinoate.

Irrespective of other components that may be present in the topical composition of the present invention, though any effective amount can be included in a final composition, the ion-pair composition can be present in an amount from 0.0001 wt % to 30.0 wt %, and in another embodiment, from 0.1 wt % to 10.0 wt %.

Other Ingredients

Genus Centipeda plant extracts, trace metals, such as copper, which can be in a skin absorbable form, and/or ion-pair compositions can be co-administered topically using any of a number of materials known in the skin care art. For example, with any of the aforementioned skin systems, typically these compositions will be administered using a suitable topical carrier. The resulting composition can be a liquid, semi-liquid, or solid preparation for application to the skin, such as a cream, a gel, a lotion, an ointment, a liniment, a paint or paste, a solution, a spray, a suspension, or a stick. Furthermore, specific skin treatments as contemplated by the present invention include, without limitation, facial and other skin scrubs, shaving creams and gels, eye/face creams and gels, make-up removers, toners, and cleansers.

Topical carriers are well known in the art, and unless incompatible with the genus Centipeda plant extract or one of the other ingredients, all are considered to be within the scope of the present invention, including conventional solvents, dispersion media, fillers, solid carriers, aqueous solutions, coatings, antibacterial and antifungal agents, isotonic and absorption delaying agents, and the like. Examples of carriers include, without limitation, emulsifiers (e.g., beeswax, stearic acid), skin protectants (e.g., dimethicone), skin conditioners (e.g., olive oil, avocado oil, cetyl stearyl alcohol), humectants (e.g., glycerin), preservatives (e.g., methyl hydroxybenzoate), solvents (e.g., water), herbal actives (e.g., aloe vera), essential oils (e.g., sandalwood oil, lavender oil), suspension agents (e.g., fractionated coconut oil), anionic emulsifying agents (e.g., cocoamidopropyl betaine), astringents (e.g., zinc phenosulfonate), antiseptics (e.g., zinc chloride), antibacterials (e.g., triclosan), healing agents (e.g., allantoin), germicides (e.g., tea tree oil), anti-inflammatories (e.g., pineapple extract), skin fresheners (e.g., lemon extract), wetting agents (e.g., cocoamidopropyl betaine), antioxidants (e.g., lecithin), skin lubricants (e.g., jojoba oil), emollients (e.g., honey), absorbing agents (e.g., purified talc), preservatives (e.g., methyl hydroxybenzoate) and/or skin/hair conditioners (e.g., olive oil).

Additional formulation ingredients that can also be utilized include rheology modifiers, examples of which include Aristoflex AVC (Ammonium AcryloyldimethyltaurateNP Copolymer), Structure Plus and Structure XL (Acrylates/Aminoacrylates/c10-30 Alkyl PEG-20 Itaconate Copolymer), Carbomer, Xanthan Gum, Carbopol ETD 2020 (Acrylate C10-30 Alkyl Acrylate Crosspolymer), Rheocin (trihydroxystearin), Hydramol PGDS (PEG-90 Diisostearate), C24-28 Alkyl Dimethicone, and Behenyl alcohol. Such a composition can also include skin feel enhancement additives such as various silicones. Examples of silicone derivations, include, without limitation, most organosilicones, organic siloxanes, and their cross polymers (e.g., dimethicone, dimethicone copolyol, cetyl dimethicone copolymer, cetyl dimethicone, stearyl dimethicone, stearoxydimethicone, behenoxydimethicone, alkyl methicone, amodimethicone, dimethicone alkyl betaine, cyclomethicone, polydimethylsiloxane, diphenyldimethyl polysiloxane, silicone elastomers, cyclomethicone and dimethicone crosspolymer, Jeesilc 6056, Dow Corning 2501). Additional skin beneficial ingredients that can be used include oil-soluble skin beneficial ingredients; water-soluble skin beneficial ingredients; hydroquinone, arbutin, hydroquinone derivatives and other skin whitening agents; dimethylaminoethanol (DMEA), alpha-lipoic acid, coenzyme Q10 (ubiquinone), carnosine, and other anti-wrinkle and anti-aging agents; vitamin C; vitamin E; water-soluble vitamin C derivatives, glycolic acid, lactic acid, mandelic acid, and hydroxy acid derivatives; and various sunscreen UVA and UVB blockers such as titanium dioxide, zinc oxide, benzophenone-3, benzophenone-4, ethylhexyl Methoxycinnamate, and such. The amounts of such ingredients are not limited to any specific limitations, as those versed in this art know that such amounts are determined by many factors that include government regulations, consumer preference, cost, marketing targets, efficacy of the composition, and the like.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

It should be noted that, where relevant, composition ingredients and the like are provided in weight percentages. Further, where applicable, color measurements were done on a Hunter Lab color meter. This color meter measures color on a scale defined as L*a*b* scale. The term “L” is from 100 to 0, representing white and black colors (lightness and darkness), respectively. A (−) value of “a” indicates a more green color. A (+) value of “a” indicates a more red color. A (−) value of “b” indicates a more blue color. A (+) value of “b” indicates a more yellow color. Various numeric values of “a” and “b” indicate degree of respective colors. Mixed colors are thus indicated by a mixed value of L*a*b* as will be noted in various examples below. The materials used had the following properties. Adenosine triphosphate disodium hydrate (molecular weight 551 Da), glutathione (molecular weight 307 Da), copper gluconate (molecular weight 453 Da, Cu=14%), copper amino acid chelate (copper 12%), fructose-1,6-diphosphate dicalcium (molecular weight 416 Da), zinc gluconate (molecular weight 455 Da, Zn=1 4%), manganese gluconate (molecular weight 445 Da, Mn=12%). The analysis of trace metals quoted in ppm in various examples, as noted below, are within +-10%.

### Example 1

### Preparation of Centipeda cunninghamii Plant Extract

Procedures for the preparation of aqueous alcohol extracts from Centipeda cunninghamii are described. These procedures are also as described in U.S. patent application Ser. No. 09/355,429, which is hereby incorporated by reference in its entirety. Other preparative methods can also be carried out as described therein, or by other methods that would be known to those skilled in the art after considering the present disclosure.

Time Method

An amount of dried plant whole is chopped into small pieces and placed into a vessel with a range of ethanol-water strengths from 10% ethanol water to 95% ethanol water, and left to macerate for a period of approximately 6 weeks. Upon completion, the material is then pressed and the resulting liquid is filtered.

Infusion Method

An amount of dried plant whole is chopped into small pieces and placed in a suitable container, then covered with boiling water and allowed to cool. The resulting material is then strained, pressed and filtered.

Decoction Method

An amount of dried plant whole is chopped into small pieces and placed into suitable container (i.e. a boiler which can have heat applied to it by different means such as gas, electricity, steam, etc.). The material is then covered with water and heated to boiling point, then left to cool, pressed and filtered.

Oil Infusion Method

An amount of dried plant whole is chopped into small pieces and placed into a suitable container and covered with either cold or heated oil. A range of oils can be used in singular or multi combination, and oils can be grapeseed, olive sunflower, evening primrose etc. The mixture is left for 1-6 weeks depending on the heat and type of oil used. A mixture of oil and ethanol can also be used. The plant is drained and pressed at the completion of the process.

Alcohol/Ethanol Extraction Method

An amount of dried plant whole is placed in a suitable vessel/container, covered with ethanol or denatured ethanol, and allowed to steep for a period of 1-6 weeks. The material is then pressed and filtered.

Examples 2-17 below describe methods for preparing compositions for use with genus Centipeda plant extract. Some examples describe preparations and calculations related to the trace metal sources that are bioavailable when administered topically. Other examples describe preparations and calculations related to the ion-pair compositions that can also be delivered topically. It is to be noted that some of the examples describe compositions that can be categorized as either a trace metal source example or an ion-pair example, as certain compositions fit in both categories. Only Examples 14-17 provide specific embodiments wherein a genus Centipeda plant extract is admixed with at least one of the trace-metal source and/or the ion-pair composition in accordance with embodiments of the present invention. One skilled in the art would clearly recognize, particularly in light of Examples 14-17 and the concentration ranges provided herein, that any one of the compositions of Example 1 (or other Centipeda extracts prepared by other methods) could be admixed and/or otherwise co-administered with any of the compositions of Examples 2-13 in accordance with the present invention. For example, in Examples where a botanical composition is described in a formulation, the Centipeda plant extract could be added to replace the botanical composition, or added in addition thereto. In examples where no botanical composition is present, then an appropriate amount as set forth herein could simply be added to the composition.

### Example 2

### Preparation of Copper ATP (CU-ATP) Solution In Situ

| Part A | |
|---|---|
| 1. Copper Gluconate | 2.25 |
| 2. Deionized Water | 97.75 |
| Part B | |
| 1. Adenosine Triphosphate (ATP) Disodium Hydrate | 2.75 |
| 2. Deionized Water | 97.25 |

Procedure: Ingredients 1 and 2 in Part “A” were mixed in a beaker. A clear blue solution was obtained. The solution had a pH of 4.0, and the color readings were L=36.15, a=−42.07, b=−6.55. These data indicate that “a” had a (−) value (green), and “b” also had a (−) value (blue), and as such, the solution was greenish blue in color. This was identified as solution, Part “A.” Ingredients 1 and 2 of Part “B” were mixed in a separate beaker. A clear, water-like solution was obtained. It had a pH of 3.1, and the color readings were L=68.32, a=−0.82, b=+0.23. Since both “a” and “b” are negligible numbers (less than 1), that indicates that the sample had no color in it. This was identified as solution Part “B.” Solutions of Part “A” and Part “B” were then mixed. A color change was immediately noted. The solution still remained clear, and no precipitate or discoloration noted. This solution was identified as solution of Cu-ATP. This Cu-ATP solution (identified as “C”) had a pH of 3.5, and the color readings were L=53.52, a=−33.58, b=4.19. The solution had a copper concentration of 1575 parts per million (ppm), or 0.1575%.

Since the Cu-ATP solution “C” obtained above had only half the amount of total copper, compared to solution Part “A”, a fresh solution of copper gluconate was obtained that contained only half the amount of total copper compared to solution Part “A”, but it still had the same amount of total copper as the solution of Cu-ATP obtained above. This fresh solution of copper gluconate was obtained by mixing 1.13 grams of copper gluconate in 98.87 grams of deionized water. The light blue clear solution thus obtained had a pH of 4.1, and the color readings were L=48.26, a=−34.28, b=−7.76. It was identified as solution “D.” A comparison of solution “C” and “D” made above shows that the “b” color reading of solution “C” had become less negative (i.e. “C” had shifted to a lesser blue color, shifting the color to a greenish blue) than that of solution “D.” This clearly confirms that copper had coordinated with ATP to form Cu-ATP complex in “C.” Same color change (i.e. turning to a more greenish blue color for sample “C”) was observed visually, as mentioned above. This confirms that the L*a*b* color readings were correlatable to visual observations. However, the L*a*b* color readings are more quantitative and measurable for exact comparisons. For this reason, the stability of Cu-ATP solution was also measured by this method.

### Example 3

### Stability of Cu-ATP Solution from Example 2

The solution “C” obtained in Example 2 was stored in a beaker with a plastic film wrapped over it. The solution was stored in full light (fluorescent lamps) under ambient room temperature conditions. The color readings were measured periodically, and any visually observed discolorations, or precipitate formations, if any, were also recorded, as noted below.

| | Initial | 1 Week | 4 Weeks |
|---|---|---|---|
| “L” | 53.52 | 51.35 | 50.54 |
| “a” | −33.58 | −35.38 | −36.08 |
| “b” | −4.19 | −5.16 | −5.56 |

### Example 4

### Preparation of Cu-ATP-Glutathione Complex In Situ

| Part A | |
|---|---|
| 1. Copper Gluconate | 2.25 |
| 2. Deionized Water | 47.75 |
| Part B | |
| 1. Adenosine Triphosphate (ATP) Disodium Hydrate | 2.75 |
| 2. Deionized Water | 47.25 |
| Part C | |
| 1. Glutathione | 1.5 |
| 2. Deionized Water | 48.5 |

Procedure: All “Part A” ingredients are mixed. A clear blue solution is obtained. All “Part B” ingredients are then mixed a separate container. A clear, water white solution is obtained. All “Part C” ingredients are mixed a separate container. A clear water white solution is obtained. The solution of “Part A” is then mixed with solution of “Part B.” A greenish blue solution is obtained, as in Experiment 1. The solution of “Part C” is added to the above mixture of solution “Part A” and “Part B,” which results in a bluish green precipitate. An analysis of this precipitate shows that both glutathione and copper to be present. The Cu content is 2100 ppm. This shows instant binding of Copper with Glutathione to form the new complex in situ.

### Example 5

### Calculation of Parts Per Million of Copper in a Composition

The parts per million (ppm) of copper content of a copper donor is calculated by: Cu ppm in Cu Donor (% Cu in Cu Donor×10,000)/100.

The Cu donor (%) needed in a composition to meet a required ppm of Cu is calculated by: % Cu donor needed=(1/Cu ppm in donor)×Cu ppm desired.

For example, a Cu donor, such as Copper amino acid chelate that has a Cu content of 20%, has the following ppm content: Cu ppm in Cu amino acid=(20×10,000)/100=2000 ppm.

To obtain a 150 ppm level of Cu in a composition, the following % of Cu amino acid chelate is needed: % Cu amino acid needed=(1/Cu ppm in Cu amino acid)×ppm desired; % Cu amino acid needed=(1/2000)×150=0.075%.

The following formula can be used for this calculation: ((63/mol.wt. of Cu source×wt. of Cu source)/total weight of composition)×1000000, in which, 63 is the atomic weight of copper, “mol. wt. of Cu source” is the molecular weight of copper “donor”, “wt. of Cu source” is the weight of copper “donor” used, “total weight of composition” is the total weight including all other additives, etc. in a composition.

To illustrate, in Example 2, molecular weight of copper gluconate is 453. If 2.25 grams of copper gluconate was used to make a 200 gram composition, identified as “C.” The copper content of “C” is: ((63/453×2.25)/200)×1000000=1564 ppm, or 0.1564%.

### Example 6

### Calculation of % Amount of a Copper Donor needed for a Specific Parts Per Million Copper Content in a Composition

The following formula can be used:
(1/ppm of Cu source)×ppm Cu desired=% Cu source needed

For example, a Cu donor, such as copper amino acid chelate with a Cu content of 20%, has 2000 ppm Cu content, as calculated above. To achieve 100 ppm of Cu in a lotion or cream product, for example, the amount of copper amino acid required is: (1/2000)×100=0.05%.

### Example 7

### Preparation of a Copper Nucleotide Facial Anti-Aging Serum with Zinc, Copper, and Manganese as Cofactor Trace Metals

| Deionized Water | 5 to 100 |
|---|---|
| Aristoflex AVC | 1.0 |
| Geogard 221 | 0.5 |
| PEG-6 | 20.0 |
| Zinc Gluconate | 0.01 |
| Copper Gluconate | 0.025 |
| Manganese Gluconate | 0.0001 |
| Adenosine Triphosphate (ATP) | 0.2 |
| Glutatbione | 0.1 |
| Fragrance | 0.15 |
| Botanical Extracts Blend | 0.25 |
| Silicone Elastomer | 5.0 |

Procedure: All copper donors (copper gluconate, zinc gluconate, and manganese gluconate) were mixed in water to give a greenish blue solution. To this solution, ATP and glutathione were added with mixing. A clear, purplish blue solution was obtained, indicating a color shift and the transfer of copper from its donors to ATP. Aristoflex AVC was then added to it and the mixture mixed for 30 minutes to form a clear greenish blue gel. All other ingredients were then added to it with mixing. A purplish blue gel was obtained. The product produced includes Zn=14 ppm, Cu=35 ppm, and Mn=0.12 ppm.

### Example 8

### Copper Glycoside Face and Body Cleanser with Different Donor Sources of Copper

| Water | to 100 |
|---|---|
| Germall II | 0.1 |
| Kathon CG | 0.06 |
| Sodium Lauryl Sulfate | 18.0 |
| Cocamidopropyl Betaine | 10.0 |
| Citric Acid | 0.15 |
| Copper Gluconate | 0.025 |
| Copper Amino Acid Chelate | 0.025 |
| Fructose-1,6-diphosphate | 0.2 |
| Fragrance | 0.5 |
| Botanical Extracts | 0.2 |

Procedure: All copper donors are dissolved in part of water (5% water) from the batch. Fructose diphosphate is then added to it with mixing to form the pre-blend. All remaining ingredients are then mixed in a separate tank. The preblend is then added to the main batch with mixing. A greenish blue syrupy cleanser product is obtained that contains 65 ppm of Cu.

### Example 9

### Copper Nucleotide and Copper Glycoside Face-Lift Mask with Ascorbic Acid and Lactic Acid as AHA and Zinc as a Cofactor Trace Metal

| PEG-6 | to 100 |
|---|---|
| Aristoflex AVC | 0.8 |
| Deionized Water | 15.0 |
| Copper Gluconate | 0.025 |
| Zinc Gluconate | 0.01 |
| Deionized Water | 1.0 |
| Adenosme Triphosphate (ATP) | 0.2 |
| Glucose Monophosphate | 0.2 |
| Ascorbic Acid | 2.0 |
| Silicone Elastomer | 10.0 |
| Chlorophenesin | 0.3 |
| Lactic Acid | 10 |

Procedure: Aristoflex is mixed with deionized water (15% portion) to a clear gel. Copper gluconate, zinc gluconate, ATP, glucose monophosphate, and water (1% portion) are mixed separately to form a light blue pre-blend solution. This is added to the main batch, and all other ingredients are also added to the main batch with mixing. A translucent light blue gel is obtained that had a copper content of 35 ppm and zinc content of 14 ppm.

### Example 10

### Trace Metals Cosmetic Gel (for Antiaging, Anti-Wrinkle, Anti-Acne, Antibacterial, and Anti-Virus Applications)

| Deionized Water | to 100 |
|---|---|
| Xanthan Gum | 1.5 |
| Glutathione | 0.15 |
| Aloe Vera powder | 0.2 |
| Dehydroacetic acid (and) | 0.5 |
| benzyl alcohol | |
| Sodium Hyaluronate | 0.1 |
| Silicone Elastomer | 4.0 |
| Polysorbate-20 | 6.0 |
| Copper Gluconate | 0.23 |
| Zinc Gluconate | 0.23 |
| ATP | 0.55 |
| Deionized Water | 5.0 |
| Glycerine | 40.0 |
| Fragrance | 0.2 |

Procedure: Deionized water is mixed with xanthan gum until hydrated. Copper gluconate, zinc gluconate, ATP, and deionized water (5.0% portion) are mixed to form a clear, light blue solution. This solution is added to the main batch and mixed. All other ingredients are then added and mixed. A light blue clear gel is obtained with copper content of 322 ppm and zinc content of 322 ppm.

### Example 11

### Trace Metal Nucleotide Shampoo for Hair Loss Reduction

| Water | to 100 |
|---|---|
| Germall II (preservative) | 0.1 |
| Kathon CG (preservative) | 0.0 |
| Sodium Lauryl Sulfate | 18.0 |
| Cocamidopropyl Betaine | 7.0 |
| Citric Acid | 0.1 |
| Copper Gluconate | 0.15 |
| ATP | 0.125 |
| Glutathione | 0.01 |
| Fragrance | 0.5 |

Procedure: All ingredients are mixed together. A clear, light blue viscous liquid is obtained which gives a high foam and cleansed hair with less hair loss when applied. The composition has a copper content of 210 ppm.

### Example 12

### Eye Gel with Copper and Zinc Fructose-1,6-Diphosphates in an Anhydrous Composition

| | |
|---|---|
| Cyclomethicone | 10.0 |
| Dimethicone | 30.0 |
| Jeesilc 3D5 | 51.8 |
| Tween-20 | 2.0 |
| Glutathione | 0.1 |
| Zinc Gluconate | 0.2 |
| Copper Gluconate | 0.2 |
| Fructose Diphosphate | 0.2 |
| PEG-6 | 5.0 |
| Geogard 221 | 0.5 |

Procedure: All ingredients are mixed together until a bluish green suspension product is obtained. The composition, prior to use, should be shaken. The composition has a copper content of 280 ppm and zinc content of 280 ppm.

### Example 13

### In Situ Preparation on Niacinamide-Ascorbate Ion-Pair

| | Column 1 | Column 2 |
|---|---|---|
| Glycerin | 20.0 | 20.0 |
| Water | 50.0 | 50.0 |
| Ascorbic Acid | 18.0 | 0.0 |
| Niacinamide (Nicotinamide) | 12.0 | 0.0 |
| Niacinamide Ascorbate | 0.0 | 30.0 |

Procedure: All of the ingredients are mixed, and the mixture is heated and stirred at 60° to 70° C. until the mixture is homogenous, e.g., for about five to ten minutes. The homogeneous mixture is cooled to room temperature, and a yellow solution is formed. It should be noted that when the composition is first mixed, as shown in Column 1, it is white in color. After preparation of the batch is complete, the product turns bright yellow, indicating the formation of niacinamide ascorbate in water solution, which is naturally yellow in color (forming the composition shown in Column 2). The color meter readings were L=91.94, a=7.21, b=22.20.

### Example 14

### Trace Metals Clear Serum (High Potency) with Centipeda cunninghamii Extract

| Ethoxydiglycol | to 100 |
|---|---|
| Propylene Glycol | 29.8 |
| Deionized Water | 20.0 |
| ATP | 5.51 |
| Copper Gluconate | 2.25 |
| Zinc Gluconate | 1.1 |
| Manganese Gluconate | 1.1 |
| Glutathione | 0.3 |
| Deionized Water | 5.0 |
| Centipeda cunninghamii | 1.0 |
| Niacinamide Ascorbate (Ion-Pair) | 5.0 |

Procedure: ATP, Copper gluconate, zinc gluconate, manganese gluconate, and deionized water (20% portion) are mixed until a clear greenish blue color is obtained (Premix A). Glutathione and deionized water (5.0 portion) are then mixed in another container until a clear solution is obtained (Premix B). Ethoxydiglycol and glycerin are mixed in a main batch tank. All other ingredients and Premix A and Premix B solutions are added to the main batch tank and mix. This batch is then filtered to remove impurities. A greenish blue viscous solution is obtained that has copper content of 3150 ppm, zinc content of 1540 ppm, and manganese content of 1320 ppm. This is used as a high potency serum for eye zone and neck zone applications to remove wrinkles and kill viruses.

### Example 15

### Preparation of a Cu-ATP Anti-Wrinkle Skin with Centipeda cunninghamii Extract

| Water | to 100 |
|---|---|
| Mineral Oil | 1.0000 |
| Phenoxyethanol | 0.9000 |
| Glycerin | 3.8000 |
| Deodorized Jojoba Oil | 0.0001 |
| Vitamin E Acetate | 0.0001 |
| Aloe Vera | 0.0001 |
| Panthenol | 0.0001 |
| Methyl Paraben | 0.2000 |
| Propyl Paraben | 0.1000 |
| PGMS-SE | 2.0000 |
| Stearic Acid | 3.0000 |
| Cetyl Alcohol | 1.2000 |
| Caustic Soda | 0.0001 |
| Deionized Water | 1.0 |
| Manganese Gluconate | 0.001 |
| Copper Amino Acid Chelate | 0.025 |
| Zinc Gluconate | 0.01 |
| Adenosine Triphosphate (ATP) | 0.2 |
| Glutathione | 0.1 |
| Fragrance | 0.6 |
| Centipeda cunninghamii | 0.65 |

Procedure: All copper donors are dissolved in water to give a clear greenish blue solution. ATP and glutathione were then added to it. The color changed to purplish blue. This solution is then added to “skin lotion base” with mixing, and all remaining ingredients were also added. A sky blue lotion was obtained. Skin lotion base was obtained by mixing all other ingredients together, then heating at b 70 to 80° C. for one hour, then cooling to ambient temperature with mixing. A white lotion is obtained which contains Cu=30 ppm, Zn=14 ppm, and Mn=1.2 ppm.

### Example 16

### Preparation of an Anti-Aging Night Cream with Trace Metal Composition, Ion-Pair Composition, and Centipeda cunninghamii Extract

| Water | to 100 |
|---|---|
| Carbomer | 0.2 |
| GMS-SE | 2.0 |
| Stearic Acid | 3.0 |
| Cetyl Alcohol | 1.5 |
| Glycerin | 1.0 |
| Jojoba Oil | 0.1 |
| Sweet Almond Oil | 0.2 |
| Sesame Oil | 0.2 |
| Apricot Kernel Oil | 0.2 |
| Panthenol | 0.0001 |
| Glydant Plus (Preservative) | 0.2 |
| Dimethicone | 2.0 |
| Vitamin E Acetate | 0.0001 |
| Vitamin A Palmitate | 0.0001 |
| Copper Adenosine Triphosphte | 0.125 |
| Niacinamide Ascorbate (Ion-Pair) | 0.15 |
| Fragrance | 0.15 |
| Centipeda cunninghamii | 0.25 |

Procedure: Copper amino acid chelate and ATP were dissolved in part of water (5% water). Fructose-1,6-diphosphate and glutathione were then added to it and the mixture stirred. It formed a precipitate of copper-ATP-glutathione and copper-fructose diphosphate-glutathione complexes. All other ingredients except fragrance and botanical extract were mixed separately and heated at 70 to 80 C, then cooled to room temperature. The trace metal complex pre-blend made above, fragrance, and botanical blends were all added to the main batch and the batch mixed. A light blue cream was obtained with copper content of 30 ppm.

### Example 17

### In Situ Reparation on Niacinamide-Ascorbate Ion-Pair with Centipeda cunninghamii Extract

| | Column 1 | Column 2 |
|---|---|---|
| Water | 65.0 | 65.0 |
| Ascorbic Acid | 18.0 | 0.0 |
| Niacinamide (Nicotinamide) | 12.0 | 0.0 |
| Centipeda cunninghamii | 5.0 | 5.0 |
| Niacinamide Ascorbate | 0.0 | 30.0 |

Procedure: All of the ingredients are mixed to form a composition that is heated and stirred at 40° to 50° C. until the mixture is homogenous, e.g., for about five to ten minutes. The homogeneous mixture is cooled to room temperature. A yellow solution is formed. It should be noted that when the composition is first mixed, as shown in Column 1, it is white in color. After preparation of the batch is complete, the product turns bright yellow, indicating the formation of niacinamide ascorbate in water solution (forming the composition shown in Column 2), which also contains Centipeda cunninghamii extract.

### Example 17

### Comparative Testing

A select group of consumers who had been users of two of the well established brands of skin products, Mary K and Beauty Control, used a skin care treatment scheme (test product) for six weeks in accordance with embodiments of the present invention. The test product included a genus Centipeda plant extract, a trace metal source, and a carrier suitable for topical administration. The consumers were asked to provide open ended comments, both their likes and dislikes of the Mary K and Beauty Control products compared to the test product administered to them over the six week period. The following tabulation shows the order in which test product were considered superior to the brands that they previously had been accustomed to using.
1. Younger looking skin.
2. More glow, with reduction of skin discoloration.
3. Reduction of Crow's Feet and facial wrinkles.
4. Smoother, softer skin (skin texture).

A minority of consumers experienced mild skin irritation at first, which is usually expected in persons having sensitive skin types when a skin treatment brand is changed. This minor irritation disappeared within a short period of time and their skin enhancement benefits continued to develop from that point forward. In no case did a consumer prefer their previous brand over skin treatment regimen administered in the present example.

It is to be understood that the above-referenced arrangements are illustrative of the application for the principles of the present invention. Numerous modifications and alternative arrangements can be devised without departing from the spirit and scope of the present invention while the present invention has been described above in connection with the exemplary embodiments(s) of the invention. It will be apparent to those of ordinary skill in the art that numerous modifications can be made without departing from the principles and concepts of the invention as set forth in the claims.

What is claimed is:

1. A cosmetic or pharmaceutical composition, comprising:
a) a genus Centipeda plant extract;
b) a trace metal source in a skin absorbing form in an amount effective for activating or enhancing superoxide dismutase enzyme; and
c) a carrier suitable for topical administration.

2. A composition as in claim 1, wherein the genus Centipeda plant extract is from the plant Centipeda cunninghamii.

3. A composition as in claim 1, wherein the trace metal source is selected from the group consisting of a copper source, a zinc source, a manganese source, and combinations thereof.

4. A composition as in claim 1, wherein the trace metal source is a copper source.

5. A composition as in claim 1, wherein the trace metal source is a zinc source.

6. A composition as in claim 1, wherein the trace metal source is a manganese source.

7. A composition as in claim 1, wherein the skin absorbing form comprises a low molecular weight transporter composition.

8. A composition as in claim 7, wherein the low molecular weight transporter composition is bound to the trace metal source, and is selected from the group consisting of nucleotides, phosphorylated saccharides, phosphorylated glycosides, and combinations thereof.

9. A composition as in claim 8, wherein the low molecular weight transporter composition is selected from the group consisting of adenosine triphosphate (ATP), adenosine diphosphate (ADP), adenosine monophosphate (AMP), flavin adenine dinucleotide (FAD), guanosine monophosphate (guanylic acid), guanosine diphosphate, inosine monophosphate (inosinic acid), inosine diphosphate, nicotinamide adenine dinucleotide (NAD), nicotinamide adenine dinucleotide reduced (NADH), and citicholine.

10. A composition as in claim 8, wherein the low molecular weight transporter composition is selected from the group consisting of glucose-1-phosphate, glucose-6-phosphate, glucose-1,6-diphosphate, fructose-1-phosphate, fructose-6-phosphate, fructose-1,6-diphosphate, sucrose phosphate, and combinations thereof.

11. A composition as in claim 1, further comprising an ion-pair delivery system including a donating composition and an accepting composition, wherein the donating composition and the accepting composition are combined to form a bound ion-pair.

12. A composition as in claim 11, wherein the donating composition is an electron donating composition and the accepting composition is an electron accepting composition.

13. A composition as in claim 11, wherein the donating composition is a proton donating composition and the accepting composition is a proton accepting composition.

14. A composition as in claim 11, wherein the ion-pair dissociates into the donating composition and the accepting composition following absorption through the skin.

15. A composition as in claim 11, wherein the ion-pair dissociates into the donating composition and the accepting composition when the ion-pair is exposed to physiological pH conditions.

16. A cosmetic or pharmaceutical composition, comprising:
a) a genus Centipeda plant extract;
b) a copper source; and
c) a carrier suitable for topical administration.

17. A composition as in claim 16, wherein the genus Centipeda plant extract is from the plant Centipeda cunninghamii.

18. A composition as in claim 16, further comprising a second trace metal source.

19. A composition as in claim 16, wherein the second trace metal source is selected from the group consisting of a zinc source, a manganese source, and combinations thereof.

20. A composition as in claim 19, wherein the second trace metal source is the zinc source.

21. A composition as in claim 19, wherein the second trace metal source is the manganese source.

22. A composition as in claim 16, wherein the copper source is in a skin absorbing form including a low molecular weight transporter.

23. A composition as in claim 22, wherein the low molecular weight transporter composition is bound to the copper source, and is selected from the group consisting of nucleotides, phosphorylated saccharides, phosphorylated glycosides, and combinations thereof.

24. A composition as in claim 23, wherein the low molecular weight transporter composition is selected from the group consisting of adenosine triphosphate (ATP), adenosine diphosphate (ADP), adenosine monophosphate (AMP), flavin adenine dinucleotide (FAD), guanosine monophosphate (guanylic acid), guanosine diphosphate, inosine monophosphate (inosinic acid), inosine diphosphate, nicotinamide adenine dinucleotide (NAD), nicotinamide adenine dinucleotide reduced (NADH), and citicholine.

25. A composition as in claim 23, wherein the low molecular weight transporter composition is selected from the group consisting of glucose-1-phosphate, glucose-6-phosphate, glucose-1,6-diphosphate, fructose-1-phosphate, fructose-6-phosphate, fructose-1,6-diphosphate, sucrose phosphate, and combinations thereof.

26. A composition as in claim 16, further comprising an ion-pair delivery system including a donating composition and an accepting composition, wherein the donating composition and the accepting composition are combined to form a bound ion-pair.

27. A composition as in claim 26, wherein the donating composition is an electron donating composition and the accepting composition is an electron accepting composition.

28. A composition as in claim 26, wherein the donating composition is a proton donating composition and the accepting composition is a proton accepting composition.

29. A composition as in claim 26, wherein the ion-pair dissociates into the donating composition and the accepting composition following absorption through the skin.

30. A composition as in claim 26, wherein the ion-pair dissociates into the donating composition and the accepting composition when the ion-pair is exposed to physiological pH conditions.

31. A cosmetic or pharmaceutical composition, comprising:
a) a genus Centipeda plant extract;
b) an ion-pair delivery system including a donating composition and an accepting composition, wherein the donating composition and the accepting composition are combined to form a bound ion-pair; and
c) a carrier suitable for topical administration.

32. A composition as in claim 31, wherein the genus Centipeda plant extract is from the plant Centipeda cunninghamii.

33. A composition as in claim 31, wherein the donating composition is an electron donating composition and the accepting composition is an electron accepting composition.

34. A composition as in claim 31, wherein the donating composition is a proton donating composition and the accepting composition is a proton accepting composition.

35. A composition as in claim 31, wherein the ion-pair dissociates into the donating composition and the accepting composition following absorption through the skin.

36. A composition as in claim 31, wherein the ion-pair dissociates into the donating composition and the accepting composition when the ion-pair is exposed to physiological pH conditions.

37. A composition as in claim 31, further comprising a trace metal source.

38. A composition as in claim 37, wherein the trace metal source is selected from the group consisting of a copper source, a zinc source, a manganese source, and combinations thereof.

39. A composition as in claim 37, wherein the trace metal source is the copper source.

40. A composition as in claim 39, further comprising a second trace metal source selected from the group consisting of a zinc source, a manganese source, and combinations thereof.

41. A composition as in claim 39, wherein the copper source provides copper in a skin absorbing form including a low molecular weight transporter composition.

42. A composition as in claim 41, wherein the low molecular weight transporter composition is bound to the copper, and is selected from the group consisting of nucleotides, phosphorylated saccharides, phosphorylated glycosides, and combinations thereof.

43. A composition as in claim 42, wherein the low molecular weight transporter composition is selected from the group consisting of adenosine triphosphate (ATP), adenosine diphosphate (ADP), adenosine monophosphate (AMP), flavin adenine dinucleotide (FAD), guanosine monophosphate (guanylic acid), guanosine diphosphate, inosine monophosphate (inosinic acid), inosine diphosphate, nicotinamide adenine dinucleotide (NAD), nicotinamide adenine dinucleotide reduced (NADH), and citicholine.

44. A composition as in claim 42, wherein the low molecular weight transporter composition is selected from the group consisting of glucose-1-phosphate, glucose-6-phosphate, glucose-1,6-diphosphate, fructose-1-phosphate, fructose-6-phosphate, fructose-1,6-diphosphate, sucrose phosphate, and combinations thereof.

45. A method of reducing or minimizing the effects of skin aging or skin damage, comprising topically applying a cosmetic or pharmaceutical composition to a skin surface, said composition, comprising:
a) a genus Centipeda plant extract;
b) a carrier suitable for topical administration; and
c) at least one additional component selected from the group consisting of:
i) a trace metal source in a skin absorbing form in an amount effective for activating or enhancing superoxide dismutase enzyme;
ii) a copper source; and
iii) an ion-pair delivery system including a donating composition and an accepting composition, wherein the donating composition and the accepting composition are combined to form a bound ion-pair.

46. A method as in claim 45, wherein the genus Centipeda plant extract is from the plant Centipeda cunninghamii.

47. A method as in claim 45, wherein the at least one additional component includes the trace metal source.

48. A method as in claim 47, wherein the trace metal source is selected from the group consisting of a copper source, a zinc source, a manganese source, and combinations thereof.

49. A method as in claim 48, wherein the trace metal source is the copper source.

50. A method as in claim 48, wherein the skin absorbing form comprises a low molecular weight transporter composition bound to the trace metal source, and is selected from the group consisting of nucleotides, phosphorylated saccharides, phosphorylated glycosides, and combinations thereof.

51. A method as in claim 45, wherein the at least one additional component includes the copper source.

52. A method as in claim 51, further comprising a second trace metal source selected from the group consisting of a zinc source, a manganese source, and combinations thereof.

53. A method as in claim 45, wherein the at least one additional component is the ion-pair delivery system.

54. A method as in claim 53, wherein the donating composition is an electron donating composition and the accepting composition is an electron accepting composition.

55. A method as in claim 53, wherein the donating composition is a proton donating composition and the accepting composition is a proton accepting composition.

56. A method as in claim 45, wherein the at least one additional component includes the trace metal source and the copper source.

57. A method as in claim 45, wherein the at least one additional component includes the trace metal source and the ion-pair delivery system.

58. A method as in claim 45, wherein the at least one additional component includes the copper source and the ion-pair delivery system.

59. A method as in claim 45, wherein the at least one additional component includes the trace metal source, the copper source, and the ion-pair delivery system.

60. A method as in claim 45, wherein the at least one additional component includes at least two components of the trace metal source, the copper source, and the ion-pair delivery system, said at least two components being associated within the composition.

61. A method as in claim 45, wherein the effects of skin aging are related to hair loss.

62. A method as in claim 45, wherein the effects of skin aging are related to skin wrinkles.

63. A method as in claim 45, wherein the effects of skin aging are related to skin discoloration.

64. A method as in claim 45, wherein the effects of skin aging are related to skin texture.

65. A method as in claim 45, wherein the effects of skin damage are resultant from a skin injury.
